(19) United States
(12) Patent Application Publication
(10) Pub. No.: US 2012/0157941 A1
(43) Pub. Date: Jun. 21, 2012

(54) Occluder to Prevent Fluid Flow Through Pump with Vacuum at Output

(76) Inventors: James Luckemeyer, San Antonio, TX (US); Kenneth Smith, San Antonio, TX (US); Christopher Brian Locke, Bournemouth (GB)

(21) Appl. No.: 13/323,223

(22) Filed: Dec. 12, 2011

Related U.S. Application Data
(60) Provisional application No. 61/423,505, filed on Dec. 15, 2010.

(51) Int. Cl. A61M 1/00; F16K 15/00

(52) U.S. Cl. 604/318; 137/511; 604/320

(57) ABSTRACT

An occluder to prevent fluid flow through a pump with a vacuum at the output is provided. Embodiments may be used in combination with negative pressure wound therapy systems, in which the occluder is coupled to a fluid source, a wound insert, and a negative pressure source, and the occluder is configured to permit fluid delivery to the wound insert under positive pressure.

### CROSS-REFERENCE TO RELATED APPLICATION

This application claims priority to U.S. Provisional Patent Application Ser. No. 61/423,505 filed Dec. 15, 2010. This provisional application is expressly incorporated by reference.

### BACKGROUND

1. Field of the Invention

Embodiments of the present invention relate generally to an apparatus and systems for restricting fluid flow through a pump with a vacuum source at the output. More particularly, embodiments of the present invention relate to an occluder for use in conjunction with fluid-instillation and negative-pressure wound therapies.

2. Background Information

Clinical studies and practice have shown that providing a reduced pressure in proximity to a tissue site augments and accelerates the growth of new tissue at the tissue site. The applications of this phenomenon are numerous, but application of reduced pressure has been particularly successful in treating wounds. This treatment (frequently referred to in the medical community as “negative pressure wound therapy,” “reduced pressure therapy,” or “vacuum therapy”) provides a number of benefits, including faster healing and increased formulation of granulation tissue. Typically, reduced pressure is applied to tissue through a wound insert (e.g., a porous pad or other manifold device). The wound insert typically contains cells or pores that are capable of distributing reduced pressure to the tissue and channeling fluids that are drawn from the tissue. The wound insert can be incorporated into a wound dressing having other components that facilitate treatment, such as, for example, a drape (e.g., adhesive surgical drape). Instillation of fluids (e.g., irrigation fluids and/or medicaments) may be used in conjunction with negative pressure wound therapy to promote healing and/or improve efficacy. One example of a system for delivering active solutions to a wound is disclosed in U.S. Pat. No. 6,398,767, incorporated herein by reference.

The negative pressure source and the fluid source may be coupled to the wound insert using a single-lumen conduit, a multi-lumen conduit with concentric lumens, or multiple conduits. A pump, such as a peristaltic pump, can be used to deliver instillation fluid to the wound insert. After a prescribed treatment period has elapsed, a negative pressure source can be configured to remove spent instillation fluids, tissue, etc. from the wound site and to apply negative pressure to the wound. An undesired consequence of coupling both fluid source and negative pressure source to the wound insert is that the negative pressure source can sometimes draw instillation fluid from the fluid bag through the peristaltic pump and into the negative pressure source.

### SUMMARY

The present disclosure includes embodiments of an occluder configured to be used with a negative pressure wound therapy system. Specific embodiments include an occluder configured to be coupled to a negative pressure source comprising a support member comprising a first channel comprising a first channel outlet and a first port; and a second channel comprising a second channel outlet and a second port; and a flexible membrane sealably coupled to the support member such that a space is formed between the membrane and the support, wherein the first port is configured to be in fluid communication with the second port through the space when positive pressure is applied to the space; and the first port is configured to be blocked when negative pressure is applied to the space.

In additional embodiments, the support member further comprises a first nipple comprising the first channel outlet; the first nipple is configured to be coupled through a conduit to a fluid pump; the support member further comprises a second nipple comprising the second channel outlet, where the second nipple is configured to be coupled through a conduit to a negative pressure source.

In certain embodiments, the support member is substantially prismatic and further comprises a first surface; the first port and the second port are disposed on the first surface; the flexible membrane is sealably coupled to the first surface; and/or the flexible membrane is ultrasonically welded to the first surface.

In specific embodiments, the support member is substantially cylindrical. In such embodiments, the support member has a central axis of symmetry, and the first channel, the first channel outlet, the first port, the second channel, the second channel outlet, and the second port are centered on the central axis; the support member further comprises at least a vent through the first channel or the second channel transverse to the central axis; and/or the support member further comprises a ball disposed within the space between the first port and the second port.

Other embodiments of an occluder configured to be coupled to a negative pressure source comprise: a rigid support member; a flexible membrane sealably coupled to the support member, wherein a space is formed between the membrane and the support member; a first aperture disposed on the support member; and a second aperture disposed on the support member, wherein the first aperture is in fluid communication with the second aperture through the space.

In specific embodiments, the rigid support member further comprises a first nipple comprising a first channel outlet, the first channel outlet in fluid communication with the first aperture; the first nipple configured to be coupled through a conduit to a fluid pump; a second nipple comprising a second channel outlet, the second channel outlet being in fluid communication with the second aperture; and/or where the second nipple is configured to be coupled through a conduit to a negative pressure source.

In certain embodiments, the rigid support member is substantially prismatic and further comprises a first surface. The first aperture and the second aperture may be disposed on the first surface. In certain embodiments, the flexible membrane is sealably coupled to the first surface; the membrane is coupled by an ultrasonic weld in some embodiments.

In some embodiments, the rigid support member is substantially cylindrical; the rigid support member has a central axis of symmetry, and/or the first aperture and the second aperture are centered on the central axis. In certain embodiments, the rigid support member further comprises at least a vent through the first channel or the second channel transverse to the central axis. Some embodiments further comprise a ball disposed within the space between the first aperture and the second aperture.

Certain embodiments of an occluder configured to be coupled to a negative pressure source comprise: a first support member comprising a first channel and a first port; a second support member comprising a second channel and a second port; and a flexible membrane sealably coupled to the first support member and the second support member such that a space is formed between the membrane and the supports, where the first port is configured to be in fluid communication with the second port when the space is under positive pressure; and a ball disposed within the space between the first port and the second port, where the ball is configured to seal one of the first port or second port when the occluder is placed under negative pressure.

In specific embodiments, the first support member further comprises a first nipple comprising a first channel outlet, the first channel outlet being in fluid communication with the first port through the first channel; the first nipple is configured to be coupled through a conduit to a fluid pump; the second support member further comprises a second nipple comprising a second channel outlet, the second channel outlet being in fluid communication with the second port through the second channel; and/or the second nipple is configured to be coupled through a conduit to a negative pressure source

In certain embodiments, the support members may have a central axis of symmetry, where the first port and second port are centered on the central axis; and/or the support members further comprise at least a vent through the first channel or the second channel transverse to the central axis.

In other aspects, a system is presented. In certain embodiments, a system for treating a wound with negative pressure therapy comprises: an occluder comprising: a rigid support comprising: a first channel comprising a first channel outlet and a first port; and a second channel comprising a second channel outlet and a second port; and a flexible membrane sealably coupled to the rigid support such that a space is formed between the membrane and the support, wherein the first port is configured to be in fluid communication with the second port through the space when positive pressure is applied to the space; a wound insert coupled to the occluder through a conduit; and a negative pressure source coupled to the wound insert through a conduit, where negative pressure is provided to the wound insert and the occluder.

In various embodiments, the system further comprises a fluid source and a pump, where the fluid source is coupled to the pump through a conduit and the pump is coupled to the occluder through a conduit; the support further comprises a first nipple comprising the first channel outlet; the first nipple is configured to be coupled through a conduit to a fluid pump; the support further comprises a second nipple comprising the second channel outlet; the second nipple is configured to be coupled through a conduit to a negative pressure source; the rigid support is substantially prismatic and further comprises a first surface; the first port and the second port are disposed on the first surface; the flexible membrane is sealably coupled to the first surface; and/or the flexible membrane is ultrasonically welded to the first surface;

In certain embodiments, the rigid support is substantially cylindrical; the rigid support has a central axis of symmetry, and where the first channel, the first channel outlet, the first port, the second channel, the second channel outlet, and the second port are centered on the central axis.; the rigid support further comprises at least a vent through the first channel or the second channel transverse to the central axis; and/or the system further comprises a ball disposed within the space between the first port and the second port.

Certain embodiments of a system for treating a wound with negative pressure wound therapy comprise an occluder comprising: support member; a flexible membrane sealably coupled to the support, wherein a space is formed between the membrane and the support; a first aperture disposed on the support member; and a second aperture disposed on the support member, wherein the first aperture is in fluid communication with the second aperture through the space; and a fluid source coupled to a pump, the pump coupled to the occluder through a first conduit in fluid communication with the first aperture.

Various embodiments of the system further comprise a wound insert coupled to the occluder through a second conduit in fluid communication with the second aperture, where the pump is configured to provide fluid from the fluid source to the wound insert; a negative pressure source coupled to the wound insert through a third conduit, where the negative pressure source is configured to apply negative pressure to the wound insert and the occluder; a first nipple comprising a first channel outlet, the first channel outlet being in fluid communication with the first aperture; the first nipple is configured to be coupled through a conduit to a fluid pump; where the support further comprises a second nipple comprising a second channel outlet, the second channel outlet being in fluid communication with the second aperture; where the second nipple is configured to be coupled through a conduit to a negative pressure source; where the support member is substantially prismatic and further comprises a first surface; where the first port and the second port are disposed on the first surface; where the flexible membrane is sealably coupled to the first surface; and/or where the flexible membrane is ultrasonically welded to the first surface.

In certain embodiments of the system, the support member is substantially cylindrical; the support member has a central axis of symmetry, and where the first aperture and the second aperture are centered on the central axis; the support member further comprises at least a vent through the first channel or the second channel transverse to the central axis; and/or further comprises a ball disposed within the space between the first aperture and the second aperture.

Various embodiments of a system treating a wound with negative pressure wound therapy comprise: an occluder comprising: a first rigid support comprising a first channel and a first port; a second rigid support comprising a second channel and a second port; and a flexible membrane sealably coupled to the first rigid support and the second rigid support such that a space is formed between the membrane and the supports, where the first port is configured to be in fluid communication with the second port when the space is under positive pressure; and a ball disposed within the space between the first port and the second port, where the ball is configured to seal one of the first port or second port when the occluder is placed under negative pressure; and a fluid source coupled to a pump, the pump coupled to the occluder through a first conduit in fluid communication with the first port.

In certain embodiments, the system further comprises a wound insert coupled to the occluder through a second conduit in fluid communication with the second port, where the pump is configured to provide fluid from the fluid source to the wound insert; a negative pressure source coupled to the wound insert through a third conduit, where the negative pressure source is configured to apply negative pressure to the wound insert and the occluder; a first nipple comprising a first channel outlet, the first channel outlet being in fluid communication with the first port through the first channel; where the first nipple is configured to be coupled through a conduit to a fluid pump; where the second rigid support further comprises a second nipple comprising a second channel outlet, the second channel outlet being in fluid communication with the second port through the second channel; where the second nipple is configured to be coupled through a conduit to a negative pressure source; where the rigid support has a central axis of symmetry, and where the first port and the second port are centered on the central axis; and/or where the rigid support further comprises at least a vent through the first channel or the second channel transverse to the central axis.

Various embodiments of the support member(s) may comprise polycarbonate or ABS plastic. Various embodiments of the flexible membrane may comprise polyethylene plastic, polyurethane, or silicone.

In certain embodiments, the occluder further comprises a sensor configured to detect whether fluid flow is permitted or occluded. The sensor may comprise electrical contacts, a pneumatic sensor, or pressure sensor. The occluder may be further configured to be coupled to a visual indicator.

Any embodiment of any of the present systems and/or methods can consist of or consist essentially of—rather than comprise/include/contain/have—any of the described steps, elements, and/or features. Thus, in any of the claims, the term “consisting of or” consisting essentially of can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 illustrates a schematic diagram of a negative pressure wound therapy system.

FIG. 2 illustrates a perspective view of one embodiment of an occluder under negative pressure.

FIG. 3 illustrates a top view of one embodiment of an occluder under negative pressure.

FIG. 4 illustrates a side section view of one embodiment of an occluder under negative pressure.

FIG. 5 illustrates a perspective view of one embodiment of an occluder under positive pressure.

FIG. 6 illustrates a top view of one embodiment of an occluder under positive pressure.

FIG. 7 illustrates a side section view of one embodiment of an occluder under positive pressure.

FIG. 8 illustrates a perspective view of one embodiment of an occluder under negative pressure.

FIG. 9 illustrates a top view of one embodiment of an occluder under negative pressure.

FIG. 10 illustrates a side section view of one embodiment of an occluder under negative pressure.

FIG. 11 illustrates a perspective view of one embodiment of an occluder under positive pressure.

FIG. 12 illustrates a top view of one embodiment of an occluder under positive pressure.

FIG. 13 illustrates a side section view of one embodiment of an occluder under positive pressure.

FIG. 14 illustrates a perspective view of one embodiment of an occluder under negative pressure.

FIG. 15 illustrates a top view of one embodiment of an occluder under negative pressure.

FIG. 16 illustrates a side section view of one embodiment of an occluder under negative pressure.

FIG. 17 illustrates a perspective view of one embodiment of an occluder under positive pressure.

FIG. 18 illustrates a top view of one embodiment of an occluder under positive pressure.

FIG. 19 illustrates a side section view of one embodiment of an occluder under positive pressure.

FIG. 20 illustrates a side section view of one embodiment of an occluder and a sensor.

FIG. 21 illustrates a side section view of one embodiment of an occluder and a sensor.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a wound-treatment method that “comprises,” “has,” “includes” or “contains” one or more steps possesses those one or more steps, but is not limited to possessing only those one or more steps. Likewise, a wound dressing that “comprises,” “has,” “includes” or “contains” one or more elements possesses those one or more elements, but is not limited to possessing only those elements. For example, in a wound dressing that comprises one of the present wound inserts and a drape, the wound dressing includes the specified elements but is not limited to having only those elements. For example, such a wound dressing could also include a connection pad configured to be coupled to a negative pressure wound therapy (NPWT) apparatus (e.g., including a vacuum source and/or a fluid source).

Further, a device or structure that is configured in a certain way is configured in at least that way, but it can also be configured in other ways than those specifically described.

Throughout this disclosure the term ‘negative pressure’ is used to refer to a pressure less than the atmospheric pressure at the particular location. For example, the application of negative pressure to a tissue site refers to the application of a pressure less than the atmospheric pressure at that tissue site. Similarly, the term ‘positive pressure’ is used to refer to a pressure greater than the atmospheric pressure at the particular location.

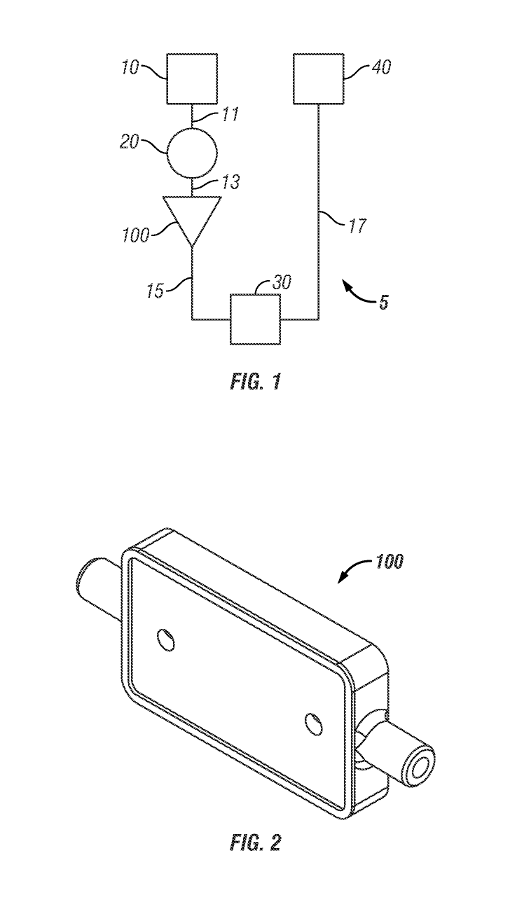

Referring now to the drawings, FIG. 1 depicts a schematic diagram of one embodiment of a negative pressure wound therapy system 5. A fluid source 10 is coupled to a pump 20 through a conduit 11. Pump 20 is coupled to an occluder 100 through a conduit 13. Occluder 100 is coupled to a wound insert 30 through conduit 15. Negative pressure source 40 is coupled to wound insert 30 through conduit 17.

Wound insert 30 is configured for placement in a wound. Pump 20 delivers instillation fluid from fluid source 10 to wound site 30 through conduits 11, 13, 15. Negative pressure source 40 is coupled to wound insert 30 through conduit 17. Conduits 11, 13, 15, 17 can comprise a single lumen conduit (e.g., switched between a vacuum source and/or a fluid source) or can comprise multiple single-lumen conduits or a multi-lumen conduit such that, for example, fluid can be delivered and/or negative pressure can be applied to wound insert 30 individually or simultaneously.

In the embodiment shown, occluder 100 is coupled to pump 20, wound insert 30, and negative pressure source 40 such that occluder 100 is between pump 20 and negative pressure source 40. When pump 20 is activated and negative pressure source 40 is deactivated, positive pressure is applied to occluder 100. Under positive upstream pressure from pump 20, occluder 100 permits fluid to flow through conduit 15 to wound insert 30.

When negative pressure source 40 is activated and pump 20 is deactivated, negative pressure is applied to occluder 100. Under negative downstream pressure from negative pressure source 40, occluder 100 prevents fluid from flowing through conduit 15 to wound 30.

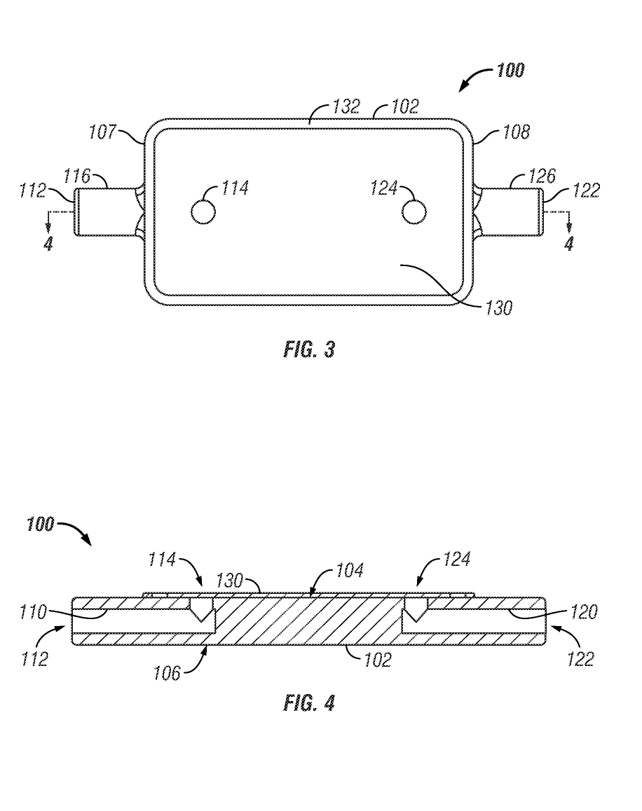

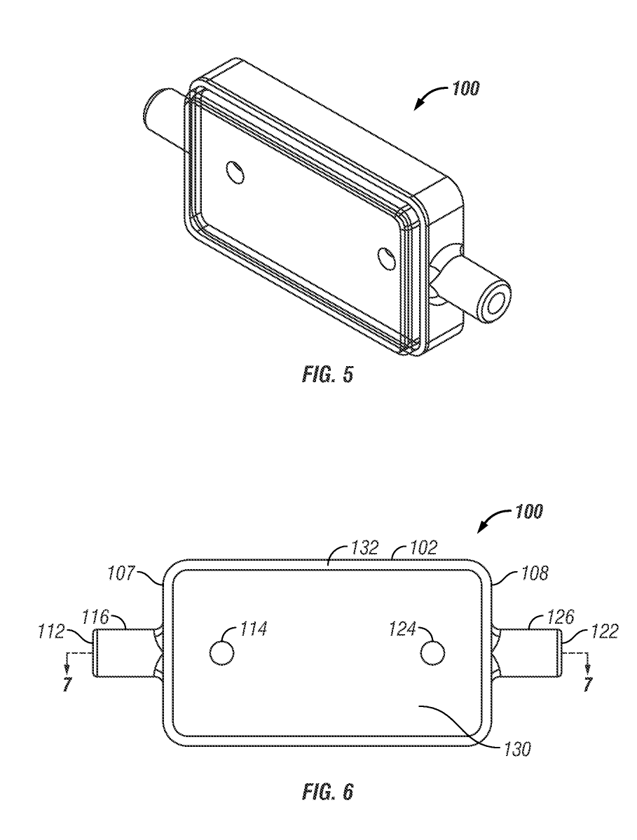

A specific embodiment of occluder 100 is shown in FIGS. 2-7. Occluder 100 is shown under negative pressure in FIGS. 2-4 and under positive pressure in FIGS. 5-7. As shown in FIG. 3, occluder 100 comprises a support member 102. In the embodiment shown, support member 102 is a prismatic rounded rectangle. In other embodiments, support member 102 may be a rectangular prism, a cylinder, a disc, or any other suitable shape.

In the illustrated embodiment, support member 102 comprises a first surface 104, a second surface 106, first side 107, and second side 108. Disposed upon first surface 104 are first port 114 and second port 124. First port 114 is in fluid communication with a first channel outlet 112 through a first channel 110. Second port 124 is in fluid communication with a second channel outlet 122 through a second channel 120. Ports 114, 124 may also be considered or called openings or apertures.

First nipple 116 is shown coupled to support member 102 on first side 107, and second nipple 126 is shown coupled to support member 102 on second side 108. First channel 110 is partially contained within first nipple 116, and second channel 120 is partially contained within second nipple 126. Nipples 116, 126 are configured to be coupled to a conduit, e.g. a multi-lumen conduit or a single-lumen conduit. In certain embodiments, nipples 116, 126 are configured to be received within the conduit, while in other embodiments, nipples 116, 126 are configured to receive the conduit. In the embodiment shown and described herein, first nipple 116 is configured to be coupled to conduit 15 coupled to fluid source 10 and pump 20, while second nipple 126 is configured to be coupled to conduit 17 coupled to negative pressure source 40.

In the illustrated embodiment, a flexible membrane 130 is sealably coupled to first surface 104 with a seal 132, creating a space 140. In this and other embodiments discussed below, flexible membrane 130 may further be resilient, i.e., capable of returning to shape after bending, stretching, or being compressed. Ports 114 and 124 are contained within space 140. Space 140 varies in volume as pressure changes within space 140. That is, space 140 increases in volume (i.e., inflates) when positive pressure is applied and decreases in volume (i.e., deflates) when negative pressure is applied.

In various embodiments, support member 102 is a rigid support, and comprises generally rigid materials such as polycarbonate, a polycarbonate blend, or ABS plastic. In other embodiments, support member 102 is comparatively more rigid than flexible membrane 130 and/or has less tendency to expand when placed under positive pressure than flexible membrane 130; in such embodiments, however, support member 102 may nonetheless be flexible (e.g., bendable).

In specific embodiments, flexible membrane 130 comprises polyethylene film or polyurethane film, though other suitable flexible, fluid-impermeable films may be used. In certain specific embodiments, flexible membrane 130 may comprise silicon tubing.

In still other embodiments support member 102 may comprise polyethylene tubing or polyurethane tubing that is thicker than flexible membrane 130.

As shown in FIGS. 2-4, when negative pressure is applied to occluder 100 (such as when negative pressure source 40 is activated to apply negative pressure through conduit 17 coupled to nipple 126) flexible membrane contracts, sealing first port 114 and second port 124. With the occluder under negative pressure, first port 114 is not in fluid communication with second port 124. Fluid is thus prevented from flowing into wound insert 30 or negative pressure source 40 in the absence of pump pressure to overcome the negative pressure seal.

As shown in FIGS. 5-7, when positive pressure is applied to occluder 100 (such as when pump 20 is activated to pump instillation fluid through conduit 15 coupled to nipple 116) flexible membrane 130 expands, placing first port 114 in fluid communication with second port 124 through space 140. In this manner, fluid may flow through first channel 110, into first port 114, into space 140, into second port 124, through channel 120, exiting occluder 100 at outlet 122.

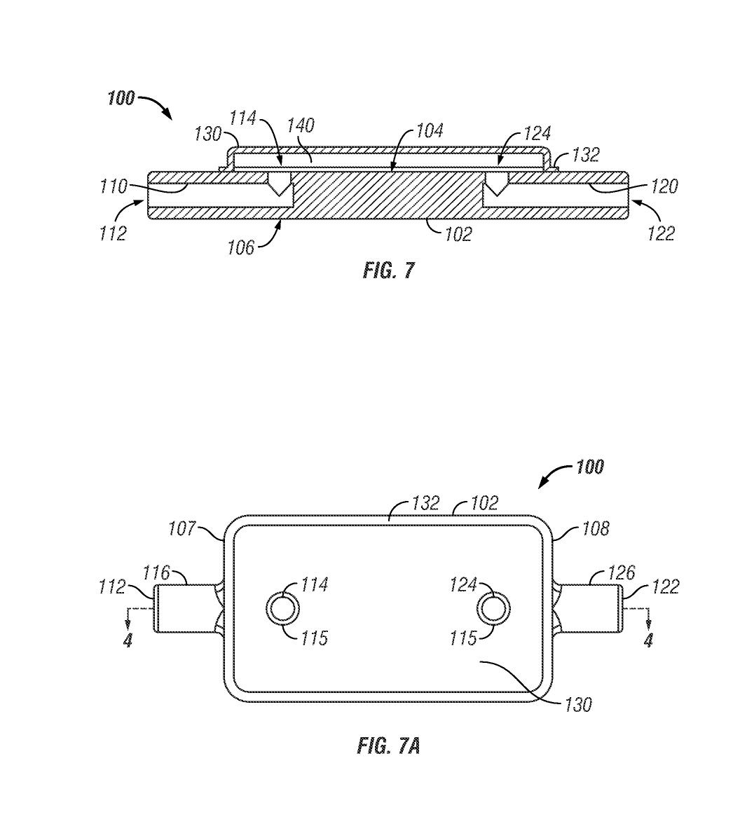

FIG. 7A illustrates a top view of an embodiment of an occluder 100, which is similar in most respects to those described in reference to FIGS. 2-7 above. However, this embodiment further comprises one or more raised ridges 115 against which flexible membrane 130 may seal. For example, as shown in FIG. 7A, occluder 100 comprises ridges 115 adjacent to and surrounding first port 114 and second port 124. Though illustrated as circles in the embodiment shown, ridges 115 may comprise any suitable shape in other embodiments to provide a raised feature against which flexible membrane 130 may form a seal. In still other embodiments, occluder 100 may comprise one or more ridges 115 running transverse (i.e., substantially perpendicular) to first nipple 116 and second nipple 126.

In certain embodiments, flexible membrane 130 is ultrasonically welded to first surface 104. Other techniques to create a seal 132 between flexible membrane 130 and first surface 104 may be used, such as liquid adhesive, transfer adhesive, or heat staking Additionally, a gasket may be used to seal flexible membrane 130 to surface 104.

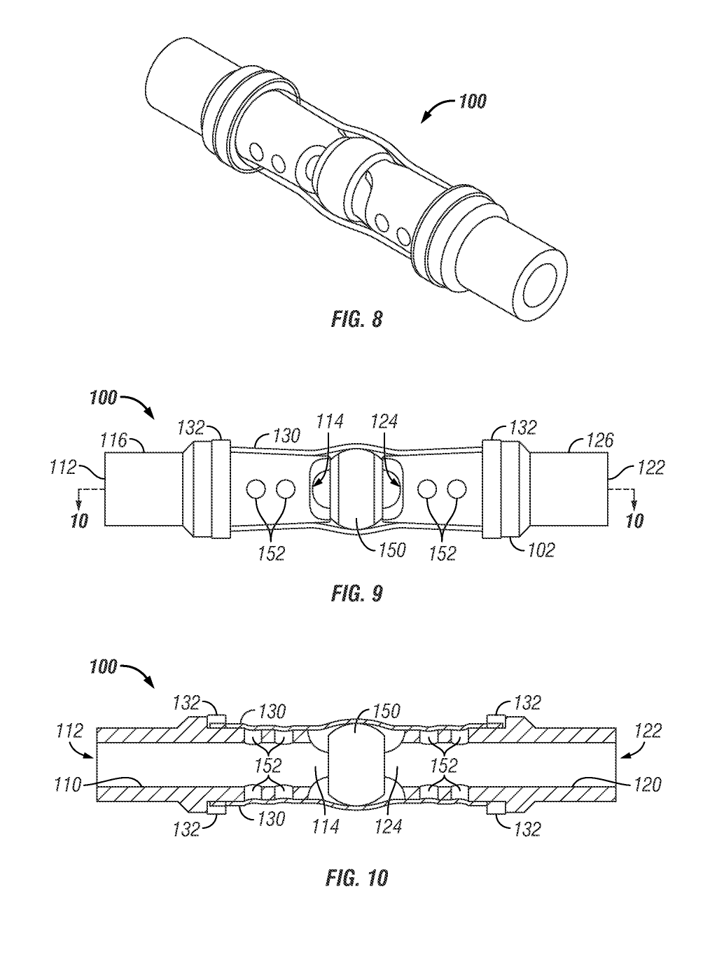

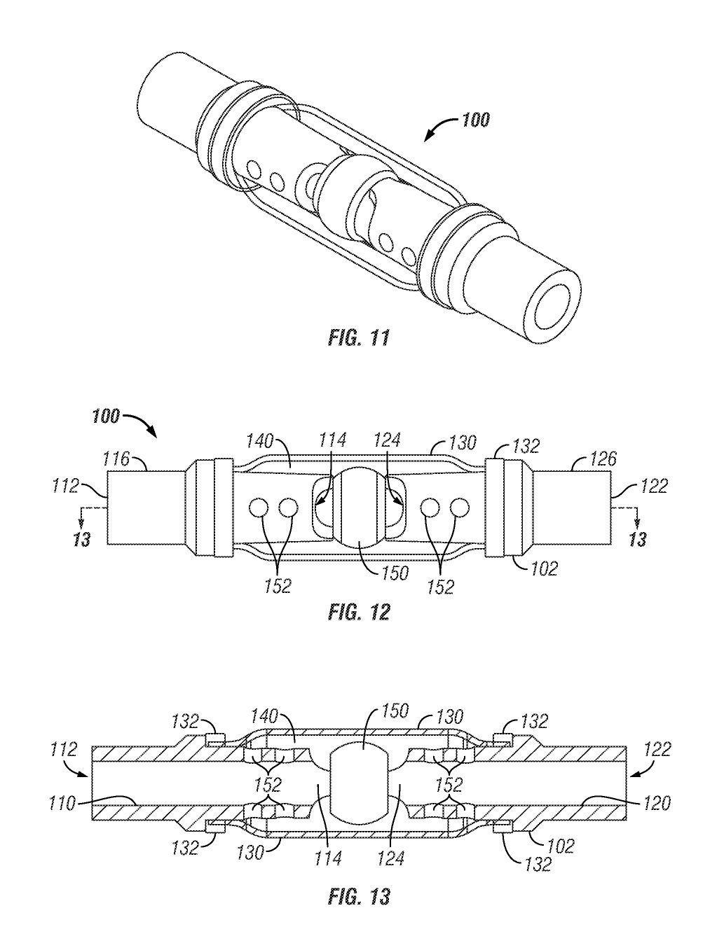

Another embodiment of occluder 100 is shown in FIGS. 8-13. Occluder 100 is shown under negative pressure in FIGS. 8-10 and under positive pressure in FIGS. 11-13. The illustrated embodiment has a central axis of symmetry running the length of occluder 100. Occluder 100 comprises rigid support structure 102, which includes a sealing bulb 150 integral with support structure 102. In the embodiment shown, support structure 102 comprises first nipple 116, channel 110, channel outlet 112, and port 114. Support structure 102 further comprises second nipple 126, channel 120, channel outlet 122, and port 124. Ports 114, 124 are adjacent to sealing bulb 150. In certain embodiments, support member 102 further comprises vents 152 transverse to channel 110.

Flexible membrane 130 is sealably coupled to support member 102 with seals 132, creating a space 140. Flexible membrane 130 is coupled to support member 102 such that ports 114, 124 and vents 152 are within space 140. In some embodiments, flexible membrane 130 is ultrasonically welded to support member 102, forming seals 132. In other embodiments, seals 132 may comprise washers or gaskets. In still other embodiments, seals 132 may be formed by coupling flexible membrane 132 to support member 102 with liquid adhesive, transfer adhesive, heat staking, or compression rings, or other suitable joining substances or joining techniques.

As shown in FIGS. 8-10, when negative pressure is applied to occluder 100 (such as when negative pressure source 40 is activated to apply negative pressure through conduit 17 coupled to nipple 126) flexible membrane contracts against sealing bulb 150, sealing first port 114 and second port 124. With the occluder under negative pressure, first port 114 is no longer in fluid communication with second port 124. Fluid is thus prevented from flowing into wound insert 30 or negative pressure source 40 when negative pressure is applied to occluder 100.

As shown in FIG. 11-13, when positive pressure is applied to occluder 100 (such as when pump 20 is activated to direct instillation fluid through conduit 15 coupled to nipple 116) flexible membrane 130 expands, placing first port 114 in fluid communication with second port 124. In this manner, fluid may flow through first channel 110, into first port 114, into space 140, into second port 124, through channel 120, exiting occluder 100 at outlet 122.

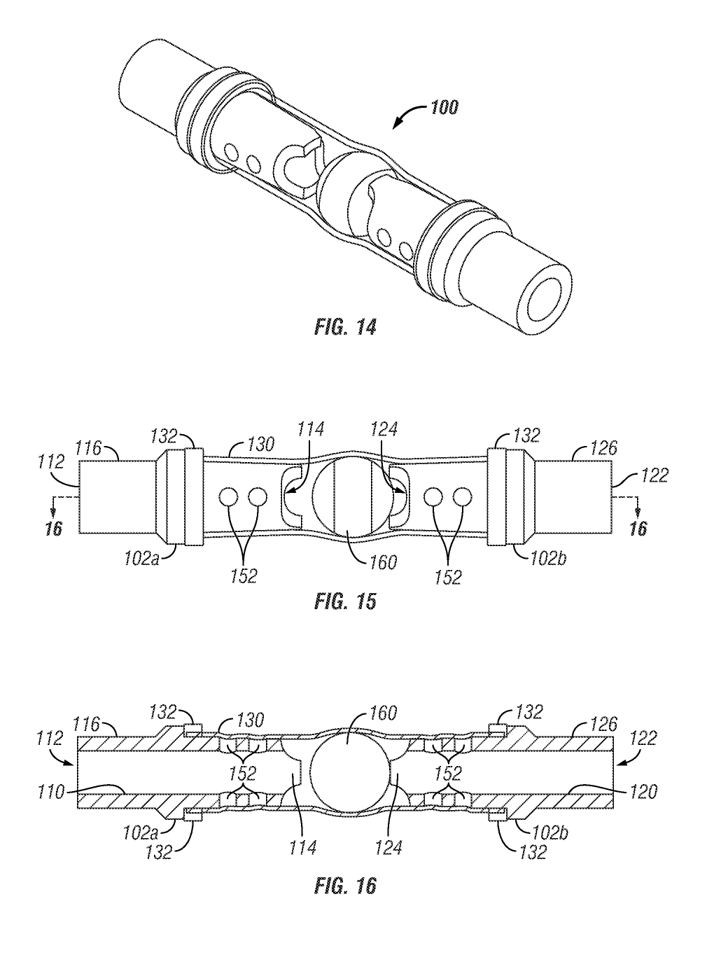

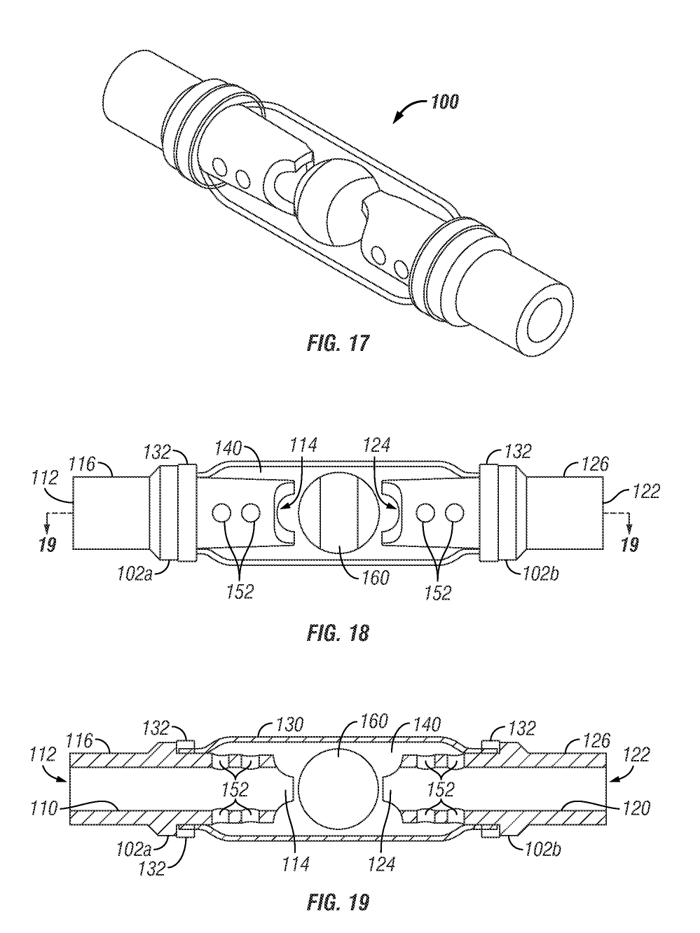

Another specific embodiment of the occluder 100 is shown in FIGS. 14-19. The embodiment is shown under negative pressure in FIGS. 14-16 and under positive pressure in FIGS. 17-19. This embodiment is similar to the embodiment disclosed in FIGS. 8-13, except that in the present embodiment, a sealing ball 160 is separate from two rigid supports 102a and 102b. In specific embodiments rigid supports 102a and 102b may be substantially identical.

The illustrated embodiment has a central axis of symmetry running the length of occluder 100. In the embodiment shown, support member 102a comprises first nipple 116, channel 110, channel outlet 112, and port 114. Support member 102b comprises second nipple 126, channel 120, channel outlet 122, and port 124. Ports 114, 124 are adjacent to a sealing ball 160. In certain embodiments, support member 102a and 102b further comprise vents 152 transverse to channel 110.

Flexible membrane 130 is sealably coupled to rigid supports 102a and 102b, creating a space 140. Flexible membrane 130 is coupled to rigid supports 102a and 102b such that ports 114, 124, vents 152, and sealing ball 160 are contained within space 140. In some embodiments, flexible membrane 130 is ultrasonically welded to support member 102. In other embodiments, seals 132 may comprise washers or gaskets. In still other embodiments, seals 132 may be formed by coupling flexible membrane 132 to support member 102 with liquid adhesive, transfer adhesive, heat staking, or compression rings, or other suitable joining substances or joining techniques. Sealing ball 160 is free to move within space 140, and is not permanently joined or affixed to either support member 102a, 102b.

As shown in FIGS. 14-16, when negative pressure is applied to occluder 100 (such as when negative pressure source 40 is activated to apply negative pressure through conduit 17 coupled to nipple 126) sealing ball is drawn toward second port 124. Flexible membrane contracts against sealing ball 160, sealing first port 114 and second port 124. With the occluder under negative pressure, first port 114 is no longer in fluid communication with second port 124. Fluid is thus prevented from flowing into wound insert 30 or negative pressure source 40 when negative pressure is applied to occluder 100. Sealing ball 160 may be spherical, ellipsoidal, cylindrical, or any other suitable shape that provides a seal to port 114 or port 124 when occluder 100 is placed under negative pressure.

As shown in FIGS. 17-19, when positive pressure is applied to occluder 100 (such as when pump 20 is activated to pump instillation fluid through conduit 15 coupled to nipple 116), flexible membrane 130 expands, placing first port 114 in fluid communication with second port 124. In this manner, fluid may flow through first channel 110, into first port 114, into space 140, around sealing ball 160, into second port 124, through channel 120, exiting occluder 100 at outlet 122.

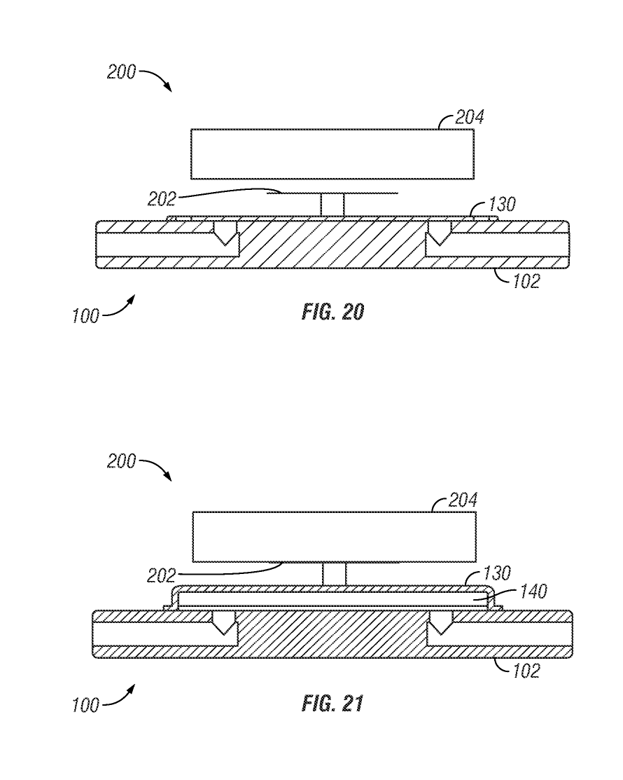

Referring now to FIGS. 20 and 21, an embodiment of occluder 100 is shown with sensor 200. In the embodiment shown, sensor 200 comprises switch 202 and detector 204. Sensor 200 is configured to send a signal indicating when fluid flow is occluded by occluder 100, and when fluid flow is permitted by occluder 100. For example, in FIG. 20, switch 202 is not in contact with detector 204; this state is known as the “off position.” When sensor 200 is in the off position, occluder 100 is occluding fluid flow. As shown in FIG. 21, switch 202 is in contact with detector 204, known as the “on position.” When sensor 200 is in the on position, occluder 100 is permitting fluid flow.

In some embodiments, sensor 200 may be an electrical sensor, with switch 202 and detector 204 comprising electrical contacts that break a circuit in the off position and complete a circuit in the on position. Sensor 200 may be configured to send a signal to a visual display indicating whether occluder 100 is in the on or off position (e.g., computer monitor, a light, etc.). Sensor 200 may also be configured to send an audible signal (e.g. a bell, buzzer, etc.) indicating whether occluder 100 is in the on or off position.

In other embodiments, sensor 200 may be a pressure sensor, a pneumatic logic sensor, visual indicator, or any other suitable sensor that may be configured to detect and indicate whether occluder 100 is permitting or occluding flow. While sensor 200 is shown in use with an embodiment of occluder 100 similar to the occluder described in FIGS. 2-7, one skilled in the art will recognize that such a sensor 200 may be used with any other occluder disclosed herein.

As will be appreciated by the reader, the principle of operation of all of the devices described herein is the same. That is the flexible member is moved by a pressure differential between the pressure internal to the device and the surrounding atmospheric pressure. When the internal pressure is less than the surrounding atmospheric pressure the resilient nature of the membrane and/or pressure differential causes the membrane to press against one or more ports of the device and thereby close the port(s). When the internal pressure is greater than the atmospheric pressure the membrane is moved away from the port(s) by that differential thereby opening the port(s).

The various illustrative embodiments of devices, systems, and methods described herein are not intended to be limited to the particular forms disclosed. Rather, they include all modifications and alternatives falling within the scope of the claims. Features of each of the embodiments may be combined with features of other embodiments.

The word ‘cylinder’ is used in a functional context to describe a longitudinally extending form having a longitudinal axis. The word is not used in its strict sense and hence does not require the part to have a constant radius along its longitudinal axis or to require a cross section to have a constant radius. For example a part may have a radius which varies along its length, and a cross section which is not circular (for example an ellipse or hexagon), but the part would fall within the meaning of cylindrical. For the avoidance of doubt, the support member of the devices shown in FIGS. 8 to 19 all fall within the meaning of cylindrical as used in this document.

The claims are not intended to include, and should not be interpreted to include, means-plus- or step-plus-function limitations, unless such a limitation is explicitly recited in a given claim using the phrase(s) “means for” or “step for,” respectively.

1. An occluder configured to be coupled to a negative pressure source comprising:
a support member comprising:
a first channel comprising a first channel outlet and a first port; and
a second channel comprising a second channel outlet and a second port; and
a flexible membrane sealably coupled to the support member such that a space is formed between the membrane and the support, wherein the first port is configured to be in fluid communication with the second port through the space when positive pressure is applied to the space; and the first port is configured to be blocked when negative pressure is applied to the space.

2. The occluder of claim 1, where the support member further comprises a first nipple comprising the first channel outlet.

3. The occluder of claim 2, where the first nipple is configured to be coupled through a conduit to a fluid pump.

4. The occluder of claim 2, where the support member further comprises a second nipple comprising the second channel outlet.

5. The occluder of claim 2, where the second nipple is configured to be coupled through a conduit to a negative pressure source.

6. The occluder of claim 1, where the support member is substantially prismatic and further comprises a first surface.

7. The occluder of claim 6, where the first port and the second port are disposed on the first surface.

8. The occluder of claim 6, where the flexible membrane is sealably coupled to the first surface.

9. The occluder of claim 8, where the flexible membrane is ultrasonically welded to the first surface.

10. The occluder of claim 1, where the support member is substantially cylindrical.

11. The occluder of claim 1, where the support member has a central axis of symmetry, and where the first channel, the first channel outlet, the first port, the second channel, the second channel outlet, and the second port are centered on the central axis.

12. The occluder of claim 11, where the support member further comprises at least a vent through the first channel or the second channel transverse to the central axis.

13. The occluder of claim 11, further comprising a ball disposed within the space between the first port and the second port.

14. The occluder of claim 1, further comprising a sensor configured to detect whether fluid flow is permitted or occluded.

15.-68. (canceled)

69. A system treating a wound with negative pressure wound therapy comprising:
an occluder comprising:
a first rigid support comprising a first channel and a first port;
a second rigid support comprising a second channel and a second port; and
a flexible membrane sealably coupled to the first rigid support and the second rigid support such that a space is formed between the membrane and the supports, where the first port is configured to be in fluid communication with the second port when the space is under positive pressure; and
a ball disposed within the space between the first port and the second port, where the ball is configured to seal one of the first port or second port when the occluder is placed under negative pressure; and
a fluid source coupled to a pump, the pump coupled to the occluder through a first conduit in fluid communication with the first port.

70. The system of claim 69, further comprising a wound insert coupled to the occluder through a second conduit in fluid communication with the second port, where the pump is configured to provide fluid from the fluid source to the wound insert.

71. The system of claim 70, further comprising a negative pressure source coupled to the wound insert through a third conduit, where the negative pressure source is configured to apply negative pressure to the wound insert and the occluder.

72. The system of claim 69, where the first rigid support further comprises a first nipple comprising a first channel outlet, the first channel outlet being in fluid communication with the first port through the first channel.

73. The system of claim 72, where the first nipple is configured to be coupled through a conduit to a fluid pump.

74. The system of claim 72, where the second rigid support further comprises a second nipple comprising a second channel outlet, the second channel outlet being in fluid communication with the second port through the second channel.

75. The system of claim 72, where the second nipple is configured to be coupled through a conduit to a negative pressure source.

76. The system of claim 69, where the rigid support has a central axis of symmetry, and where the first port and the second port are centered on the central axis.

77. The system of claim 76, where the rigid support further comprises at least a vent through the first channel or the second channel transverse to the central axis.

78. The system of claim 69, further comprising a sensor configured to detect whether fluid flow is permitted or occluded.

79. The system of claim 78, further comprising a visual indicator coupled to the sensor.
